(19) United States
(12) Patent Application Publication
(10) Pub. No.: US 2025/0366733 A1
(43) Pub. Date: Dec. 4, 2025

(54) SYSTEM AND METHOD FOR DETECTING EQUINE ASTHMA

(71) Applicant: Trustees of Tufts College, Medford, MA (US)

(72) Inventors: Melissa R. Mazan, Medford, MA (US); Thomas Coons, Medford, MA (US); Christopher Simotas, Medford, MA (US); Brian Barrows, Medford, MA (US)

(21) Appl. No.: 18/864,283

(22) PCT Filed: May 10, 2023
(86) PCT No.: PCT/US2023/066822

§ 371 Date: Nov. 8, 2024

Related U.S. Application Data
(60) Provisional application No. 63/340,236, filed on May 10, 2022.

(51) Int. Cl. A61B 5/087; A61B 5/00

(52) U.S. Cl. CPC A61B 5/087; A61B 5/682; A61B 5/7275; A61B 2503/40; A61B 2562/0247

(57) ABSTRACT

Systems and methods are provided for determining whether a horse has equine asthma. A system that provides a single airflow passage for the horse as the horse breathes. The system is operated to obstruct the single airflow passage as the horse continues to perform breathing motions. The pressure about a muzzle of the horse is measured while the single airflow passage is obstructed, and the horse continues to breathe. The pressure about the muzzle of the horse is analyzed to determine that the pressure about a muzzle of the horse while the single airflow passage is obstructed, and the horse continues to make breathing motions is indicative of equine asthma. A report can be generated indicating that the horse is suffering from equine asthma based on the analysis.

### CROSS-REFERENCE TO RELATED APPLICATIONS

This application claims the benefit of U.S. Provisional Application No. 63/340,236, filed on May 10, 2022. The contents of the application is incorporated by reference herein.

### STATEMENT REGARDING FEDERALLY SPONSORED RESEARCH

N/A

### BACKGROUND

Equine asthma is an important cause of poor performance for high-performance horses, second only to lameness. Furthermore, equine asthma can have substantial implications for quality-of-life for older horses with chronic disease who can become essentially respiratory cripples. Equine asthma is often a silent condition, particularly in young or otherwise healthy horses, and in some cases does not manifest symptoms other than slower running times. For performance horses, which can win or lose races by hundredths of a second, undiagnosed equine asthma can have substantial financial repercussions.

Unfortunately, diagnosing equine asthma can be very difficult. In human medicine, lung function and a diagnosis of asthma can be performed using portable tests known as “peak flow meters.” The “peak flow meter” measures how much air is coming into and going out of the human's lungs. However, to use a “peak flow meter,” the person being tested must take a large breath in and exhale forcefully until their lungs are empty. Of course, it is not possible to, on command, have a horse follow this process of filling its lungs and then forcefully exhaling until its lungs are empty. Thus, elaborate systems have been designed to attempt to assess whether a horse has asthma without necessitating careful controls of the horse's breathing.

Resistance for fluid flow can be defined as a difference in pressure divided by a difference in flow, as shown in equation 1. Accordingly, resistance can have units of measurement such as, for example, cmH₂O per liters per second.

$$Resistance = \frac{Δ Pressure}{Δ Flow} \qquad ( 1 )$$

Some conventional systems for measuring resistance in mechanical lung function of a horse can include directly measuring a change in volume of a lung and deriving a change in pressure from the volume change, according to known gas laws. In these conventional systems, the pressure differential can be a maximum change in thoracic esophageal pressure during either inspiration or expiration and change in volume can be measured as a change in flow during the measured pressure change. The thoracic esophageal pressure may be measured as a proxy for alveolar pressure, as direct measurement of alveolar pressure is too invasive for most species. Measuring an esophageal pressure, however, requires that a horse first swallow an esophageal balloon. A horse may be unwilling to swallow the balloon and may further develop a bloody nose as a result of the procedure. Consequently, this method may not be practical, and in fact, any resulting nose bleeding can restrict the airways of a horse, thus negatively impacting athletic performance, for example. Other conventional systems can measure resistance through forcing sinusoidal pulsations of compressed air into a lung of a horse, and observing the response (e.g., forced oscillatory mechanics), but these systems can require specialized equipment that may only be available, for example, at special facilities, such as research institutions or equine hospitals. Additional methods for measuring a pressure to calculate a resistance may include measuring a change in diameter of a horse's chest during breathing, however, this method can be unreliable, and can also require specialized equipment that is not easily portable and may be available only at certain locations.

Thus, it would be desirable to have systems and methods for assessing equine asthma that do not require the horse to control its breathing in a particular way or require access to large and complicated systems for assessing the horse's lung function under normal breathing conditions.

### SUMMARY

The present disclosure provides systems and methods that overcome the aforementioned drawbacks by providing systems and methods for detecting equine asthma.

In accordance with one aspect of the disclosure, a system is provided for detecting equine asthma in a horse. The system includes a mask configured to enclose a portion of a muzzle of a horse, an airflow path from the mask, and a shutter configured to obstruct the airflow path from the mask. The system also includes at least one sensor configured to measure at least one of air pressure or airflow before the shutter obstructs the airflow path and to measure at least one of air pressure or airflow after the shutter obstructs the airflow path. The system further includes a processor configured to receive feedback from the at least one sensor, determine a pressure in the mask after the shutter obstructs the airflow path from the mask, and generate a report indicating an equine asthma condition of the horse.

In accordance with another aspect of the disclosure, a system is provided for detecting equine asthma in a horse. The system includes a mask including a top portion to partially receive a nose of a horse and a bottom portion to partially receive a mouth of the horse, the mask defining a mask chamber. A tube protrudes from the top portion of the mask, the tube including a first opening in fluid communication with the mask chamber and a second opening in fluid communication with an ambient air. A pressure sensor is disposed at a first position within the tube, the pressure sensor being configured to measure an air pressure within the tube. A flowmeter is disposed at a second position within the tube, the flowmeter being configured to measure an air flow through the tube. An interrupt assembly includes an actuator and a shutter. The actuator is configured to move the shutter between: a first position, in which the shutter does not extend into the tube, and a second position in which the shutter obstructs an airflow path through the tube. In the second position, the shutter is positioned between the pressure sensor and the flowmeter. A processor configured to receive, from the flowmeter, a first flow rate measurement; receive, from the pressure sensor, a first pressure measurement; determine, based at least in part on the first flow rate measurement and the first pressure measurement, an equine asthma condition of the horse; and output to one of a display or a memory, the equine asthma condition.

In accordance with another aspect of the disclosure, a method is provided for determining whether a horse has equine asthma. The method includes engaging the horse with a system that provides a single airflow passage for the horse as the horse breathes and measuring a rate of air flow through the single airflow passage and an ambient air pressure at a first time. The method also includes obstructing the single airflow passage as the horse continues to breathe and measuring a pressure about a muzzle of the horse at a second time while the single airflow passage is obstructed and the horse continues to breathe. The pressure about the muzzle of the horse, the rate of air flow, and the ambient air pressure are analyzed to determine that the pressure about the muzzle of the horse while the single airflow passage is obstructed and the horse continues breathing motions is indicative of equine asthma. A report is generated indicating that the horse is suffering from equine asthma.

The foregoing and other aspects and advantages of the invention will appear in the following description. In the description, reference is made to the accompanying drawings that form a part hereof There is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic of an equine asthma detection device in accordance with the present disclosure.

FIG. 2 is a flowchart setting forth some non-limiting steps of a method for assessing a horse for equine asthma, which may be performed using the system of FIG. 1.

FIG. 3 is a schematic block diagram of the system of FIG. 1 illustrating one, non-limiting configuration.

FIG. 4 is a partial schematic view of the equine asthma detection device shown in FIG. 1.

FIG. 5 is a graph of a pressure measured before and after introduction of an occlusion in an airway generated in accordance with the present disclosure.

### DETAILED DESCRIPTION

Horses can be kept for an athletic purpose, for example, racing, riding, jumping, etc. Equine asthma is a condition that can negatively impact a horse's quality of life, athletic performance, and economic value to an owner. Mild cases of asthma may not produce observable symptoms (e.g., cough or nasal discharge), and can thus go undetected or undiagnosed. However, even where asthma does not produce notable symptoms in a horse, it can still produce an adverse consequence for the horse or for an owner of the horse. For example, horses can be kept for racing, and their economic value may correlate to a speed of the horse, or the horse's ability to win races. In some instances, horse races can be decided by tenths, or even hundredths of a second, and thus, even a minimal impact to a horse's performance due to poor oxygenation can negatively affect the outcome of a race, and can thus produce a significant decrease in a value of a horse.

In addition to causing a decrease in performance of a horse, mild asthma can develop into more severe cases that can manifest visible symptoms and can ultimately render a horse unfit for athletic activity. Further, the primary driver of equine asthma is the barn environment, which can necessarily expose a horse to toxic dust and molds. Removing a horse from an environment productive of equine asthma may not be a viable treatment or prevention option. Thus, it can be of critical importance to detect asthma in a horse while the asthma may be treated, managed, or reversed, and before it produces a long-term harm to the horse.

As previously stated, mild cases of equine asthma may not produce visible symptoms and can evade detection even in clinical examinations of an equine athlete. Conventionally, a diagnosis for equine asthma can be based on medical history, physical examination, and examination of cytology obtained by bronchoalveolar lavage, which is an invasive method of obtaining cells from the lower airways of the horse. In other examples, a diagnosis for equine asthma can use esophageal balloon pneumotachography (EB/P), which requires placement of an esophageal balloon along an esophagus of a horse and measuring volume changes in the esophageal balloon. Forced oscillatory mechanics (FOM) is another example of obtaining a diagnosis for equine asthma. However, FOM may require use of equipment that is only available at certain facilities or lacks portability, which can make it unsuitable for use in ambulatory medicine. In some cases, a diagnosis based on these methods and data points may be inaccurate, may be unduly invasive, or may fail to detect mild cases of equine asthma.

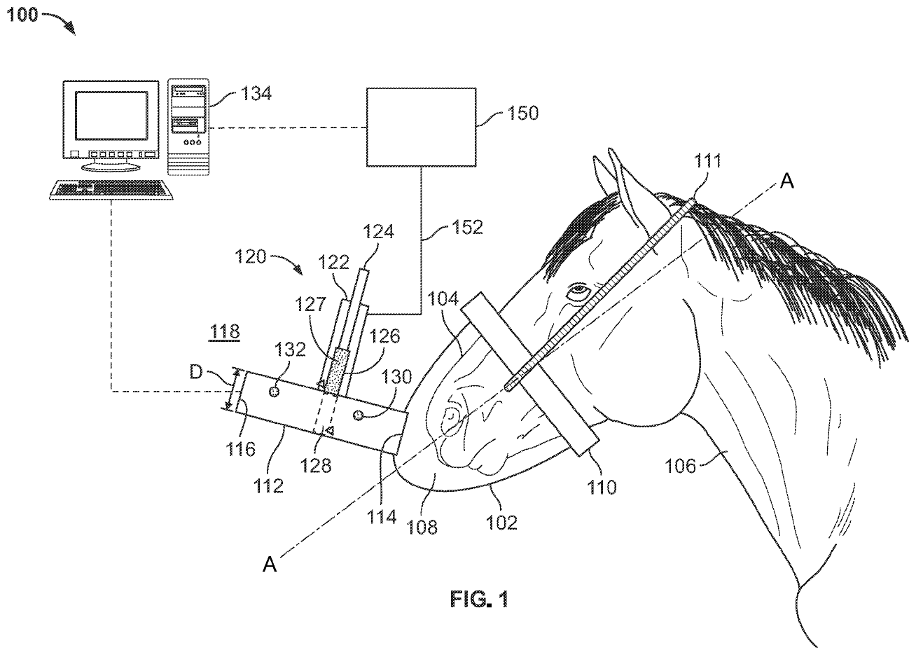

Referring to FIG. 1, a system can be provided for measuring an airway resistance of a horse and can include mechanisms for measuring a pressure differential and a flow differential. In some configurations, the system may be lightweight and/or portable. In this respect, FIG. 1 illustrates one non-limiting example of equine asthma detection device 100. As illustrated, the device 100 includes a mask 102 that can be sized and configured to fit over a muzzle 104 of a horse 106. The mask 102 can include a chamber 108 which is specifically designed and configured for the horse's muzzle 104 to be received. The mask 102 includes a seal 110, which can extend around a circumference of the muzzle 104 and is specifically designed to form a substantial seal therewith despite the muzzle 104 being covered in hair or being of a particular size. Thus, the seal 110 is designed to control against air exiting the mask chamber 108 at the interface between mask 102 and the horse's muzzle 104. In some configurations, the seal 110 may be designed to be completely airtight. In other configurations, the seal may be substantially airtight. In the latter case, the system may be able to tolerate a leak of, for example, 1% of peak flow. In this case, substantially airtight may include a leak of 1% or less. In one, non-limiting example, peak expiratory flow may be 4.5 liters, or 4500 mls, in which case a leak of 0.045 liters or 45 mls or less can be tolerated and detected by the device 100. In some configurations, a strap 111 can be provided to secure the mask 102 to the horse 106. The strap 111 may be used to tighten the mask 102 against the muzzle 104 to ensure the desired amount of control against airflow by the seal 110. Additionally or alternatively, a variety of sizes of masks 102 may be provided to accommodate different horse sizes and/or breeds.

The mask 102 can extend in three dimensions and be formed from a lightweight material that allows a horse to support the mask. The mask 102 may be printed or otherwise formed by traditional manufacturing processes, such as molding. In some configurations, a profile of the mask 102 may not be circular but may instead conform to a general profile of a horse's muzzle 104. For example, the mask 202 may not be symmetrical about axis A, and a top of the mask 102 (e.g., the portion above axis A) can be shaped to conform to the top of the horse's muzzle 104, while a bottom of the mask 102 (e.g., the portion beneath axis A) can be shaped to conform to a bottom of the horse's muzzle 104. In some configurations, the mask 102 may be fully or partially transparent to provide a view of the horse 106 through the mask 102.

In some configurations, the seal 110 can also be printed. Regardless of the particular manufacturing process uses, the seal 110 can include a flexible diaphragm. In some configurations, the seal 110 can be integrally formed with the mask 102.

In some configurations, a pipe 112 extends from the mask 102 to provide an air flow path from the mask 102. The pipe 112 may be coupled to the mask 102, with a first open end 114 fluidly connected to the mask chamber 108, and a second open end 116 in fluid communication with the ambient air 118. Thus, the horse 106 can exhale into the mask chamber 108 and exhaled air can flow into the first open end 114, through the pipe 112, and out the second open end 116. An air flow for inhalation or inspiration can follow the reverse of that sequence (e.g., as the horse 106 inhales, ambient air 118 can be drawn into the second open end, through the pipe 112, and into the mask chamber 108 through the first open end 114). A diameter, D, of the pipe 112 can be selected based on an anticipated rate of air flow through the pipe 112, an equine characteristic, or other parameters. In one non-limiting example, the diameter D may be at least as large as the diameter of a trachea of the horse 106, so that the pipe 112 itself does not impede a flow of air by introducing a constriction in the air flow. In some configurations, the diameter D of the pipe 112 can be at least about 6 cm, or at least about 7 cm. The diameter D can be larger or smaller, based on the particular horse that is being measured and, thus, smaller sizes of the mask 102 may include smaller diameters of the pipe 112 integrated therewith. Alternatively, one pipe 112 may be configured to be engaged with any of a variety of masks 102 and mask sizes. For example, a mask can include a uniform attachment interface that can engage a corresponding interface of pipes having a variety of diameters to facilitate an installation of differently sized pipes on a mask.

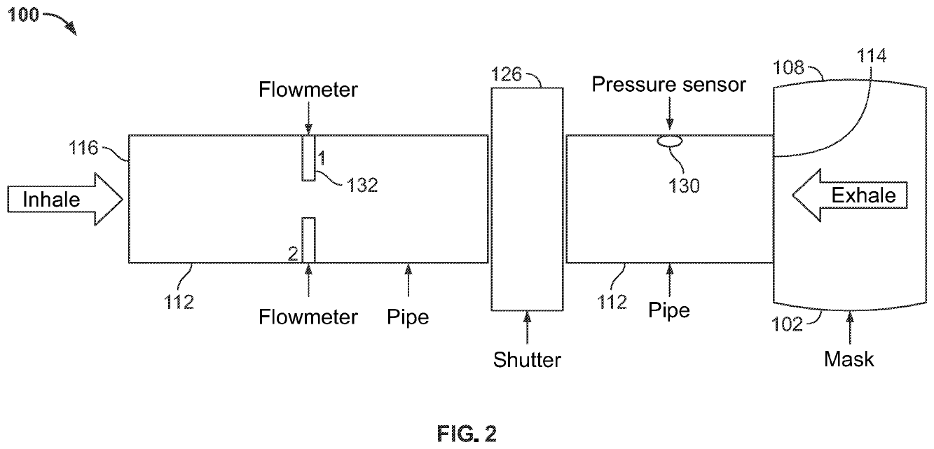

The pipe 112 can be positioned on the mask 102 to advantageously complement the anatomy and physiology of the horse 106. Indeed, as horses are obligate nose breathers, without the option of breathing through the mouth, a flow path of an equine asthma detection device may be positioned away from the mouth and closer to the nose. In one non-limiting example, the pipe 112 may be in approximate alignment with the nostrils of a horse 106 so that flow from the nostrils through the pipe 112 has minimal interruption or encounters minimal resistance in flowing from the nostrils of the horse 106 through the pipe 112. For example, as shown in the non-limiting example in FIG. 2, the pipe 112 can be positioned on an upper portion of the mask 102, above the central axis A of the mask 102. In some non-limiting examples, the pipe 112 can be substantially parallel with axis A, while in other examples, including as shown, the pipe 112 can be positioned at an oblique angle relative to the axis A. In some cases, an angle of a pipe (e.g., pipe 112) can be adjustable by an operator to be adapted to a particular horse. For example, a mask can include mechanical features (e.g., hinges) to allow rotational movement of the pipe 112.

Referring back to FIG. 1, an interrupt assembly 120 can be provided along the tube 112 of the equine asthma detection device 100. The interrupt assembly 120 can include a housing 122, an actuator 124, and a shutter 126. The shutter 126 may form a valve. The valve may include ball, butterfly, diaphragm, gate, pinch, piston, or plug valve. In the non-limiting example shown in FIG. 1, the shutter 126 is shown in a first position 127, where it does not extend into airflow between open ends 114 and 116 of the pipe 112. A second position 128 is illustrated to which the shutter 126 can be moved to block flow between the open ends 114, 116, and thus allow for a pressure in the mask chamber 108 to achieve equilibrium with the alveolar pressure in the lungs of the horse.

In operation, a signal may be provided to the actuator 124 to selectively move the shutter 126 between the first position 127 and the second position 128. In some configurations, the actuator 124 can be pneumatically driven. In some configurations, the actuator 124 may comprise a solenoid. The solenoid may be electrically driven. The interrupt assembly 120 can be configured to move the shutter 126 from one position to the other in less than a time that corresponds to the horse's reaction speed to a change in fluid pressure within the mask 102 caused with the shutter 126 closes. In one non-limiting example, the interrupt assembly 120 may be designed to close the shutter 126 in, for example, less than 50 ms, or less than 20 ms, or less than 10 ms. The shutter 126 may default to being in the first or open position, allowing air flow through the pipe 112. For example, a spring (not shown) may be provided in the interrupt assembly 120 which can mechanically bias the shutter to default to either the first position 127 or the second position 128. In some cases, it can be advantageous to configure the interrupt assembly to default to the first position 127, as can allow air flow through the mask in the absence of power to the interrupt assembly.

In some cases, the actuator can be in communication with a power source 150, which can provide a power for selectively moving the shutter 126 between the first position 127 and the second position 128. In some cases, the power source can be separate from the mask 102 and can be connected to the mask through a conduit 152. In some case, it can be advantageous to provide a power source 150 separate from the mask 102, as a weight of the power source could otherwise increase a difficulty of supporting the mask 102 relative to the horse 106. In some cases, for example, the power source 150 can include cannisters of compressed air. In some examples, cannister of compressed air used for powering the actuator 124 can have an empty weight of about 2.5 pounds and can define a volume of at least 45 cubic inches. In some examples, any known cannisters or reservoirs for compressed air (e.g., including compressed air tanks) can be used to power the actuator 124. The conduit 152 can be an air hose which can transfer a compressed air to the interrupt assembly 120 to selectively move the shutter 126 between the first and second positions 127, 128. In some cases, using cannisters of compressed air as a power source can advantageously improve a portability of a system for detecting equine asthma, as a power source containing one or more cannisters of compressed air can be easily carried to a barn environment. In some cases, a single test for a detecting an equine asthma in a horse can require 24 valve closure operations (e.g., 24 instances of moving the shutter 126 between the first position 127 and the second position 128). In some cases, a cannister of compressed air having a volume of 45 cubic inches can provide a power to close a valve having a diameter of 3.5 inches in 17 ms or less. In some cases, a single cannister of compressed air having a volume of 45 cubic inches can provide a power sufficient to test eight horses for equine asthma (e.g., to perform 192 valve closure operations). In other examples, larger or smaller compressed air cannisters can be used in a power source 150, as can provide higher or lesser capacity respectively. Further, a power source can include more than one cannisters of compressed air, which can increase a power capacity of the power source, allowing for a greater number of tests to be performed on a single charge of the power source. In other non-limiting examples, the power source 150 can include cannisters of compressed carbon dioxide. In some non-limiting examples, the power source can be an electrical power source (e.g., a battery, or a wired connection to an AC power source), and the conduit 152 can be an electrical wire to provide a signal from the electrical power source 150 to the interrupt assembly 120. Other configurations are possible, and an interrupt assembly can receive a power to selectively move a shutter using any know systems for powering valves or shutters.

In some configurations, a first sensor, which may be a pressure sensor 130, can be provided, which can sense a pressure along the flow path from the mask 102 of the equine asthma detection device 100. For example, FIG. 1 illustrates the pressure sensor 130 positioned between the first open end 114 and the interrupt assembly 120. In this way, before the shutter 126 is in the first position 127, the pressure sensor 130 can sense an ambient pressure, as the section of the pipe 112 is in fluid communication with the ambient air 118. Correspondingly, when the shutter 126 is in the second position 128, blocking fluid communication between the mask chamber 108 and the ambient air 118, the pressure sensor 130 can detect a pressure in the mask 102 (i.e., a pressure within the mask chamber 108), which can be approximately equal to the alveolar pressure of the horse. As explained above, the interrupt assembly 120 can be configured to move the shutter 126 from one position to the other in less than a time that corresponds to the horse's reaction speed to a change in fluid pressure within the mask 102 caused with the shutter 126 closes. In this way, the horse 106 will not have time to react and change its breathing pattern. A quick valve closure (e.g., a time for the shutter 126 to move from the first position 127 to the second position 128) can further limit an undesired amount of air to pass from the mask chamber 108 to the ambient air 118 once the interrupt assembly 120 starts to move the shutter 126 to the second, closed position 128. In some examples, the interrupt assembly 120 can be configured to close a valve (e.g., move the shutter 126 to the second position 128) in less than 20 ms. In some examples, including, for example, when the power source 150 includes compressed air cannisters, as described above, the interrupt assembly can be configured to close the valve in less than 17 ms.

In one, non-limiting example, an interrupt interval may be utilized between the time when the shutter 126 begins to move to the second position 128 and when breathing for the horse 106 will have been interrupted. During interruption of the breathing of the horse, the horse can be unaware of the interruption, and can continue breathing motions. As a further non-limiting example, a time of 150-200 ms may be selected for a breath interruption time, as this is the amount of time that it will take for alveolar pressure to equilibrate with mask pressure. That is, once the shutter 126 begins to move to the second position 128, the pressure in the chamber 108 in the mask 102 will readily increase as the horse continues to exhale until it reaches the alveolar pressure of the horse 106. Thus, the pressor sensor 130 will detect the alveolar pressure of the horse 106. In some examples, an interrupt interval can be configured by an operator of the interrupt assembly, including as can be adapted to horses of different sizes or types.

In one, non-limiting example, peak flow through the pipe 112 may be 4.5 liters/s or 4500 mls/1000 msec. As noted above, the shutter 126 may be configured to close in approximately 20 ms. Thus, peak flow over the approximately 20 ms that the shutter is closing would result in a loss of less than 90 mls of air from the mask chamber 102. An average tidal volume for a horse can be approximately 8 liters, so this would represent a loss of 1% of volume, which yields accurate measurements with the device 100 to determine equine asthma. In some examples, the interrupt assembly can provide an air loss (e.g., a flow of air through the open end 116 from the beginning of the shutter 126 closure to when the shutter 126 reaches the second position 128) of less than 5% of a tidal volume for a given horse, while providing accurate measurements for resistance.

A second sensor, which may be a flow sensor 132 can be included in the device 100. The flow sensor 132 can be positioned along an air flow path of the pipe 112 proximate to the second open end 216. With this design, the flow sensor 132 can provide a flow measurement immediately before the shutter 126 is moved to the second position 128, obstructing flow through the pipe 112. This flow measurement can be used in the calculation of the airway resistance of the horse 106, as will be described.

In some configurations, the flow sensor 132 can be a Fleish #5 pneumotach. In other configurations, the flow sensor 132 can be a pitot tube device, an ultrasonic flow measurement device, an anemometer, a Venturi device, or a magnetic flow meter. The flow sensor 132 can be configured to detect flow rates of, for example, at least between 3 liters per second and 25 liters per second, which can allow the flow sensor 132 to measure flow rates for horses having mild, moderate, or even severe asthma.

Even well-trained and well-handled horses resent and can react suddenly and unpredictably to equipment that they perceive to be heavy or burdensome, which can become dangerous both for the horse and the operator. Thus, the above-described devices 100 are sufficiently light and can be easily placed on and equally easily taken off the horse in the case of the animal ‘spooking’. In one example, a total weight of the apparatus for a mask (e.g., a combined weight of the mask 102, interrupt assembly 120, and pipe 112) can be about 3.5 lbs. In some cases, a total weight for the apparatus for the mask can be between about 2 to 5 lbs., between about 3 to four lbs., or other weights that can be jointly supported by a head of a horse and the horse's handler. As most veterinarians travel to the barn to perform examinations rather than the horse traveling to the veterinarian, the testing equipment must also be portable and easy to use.

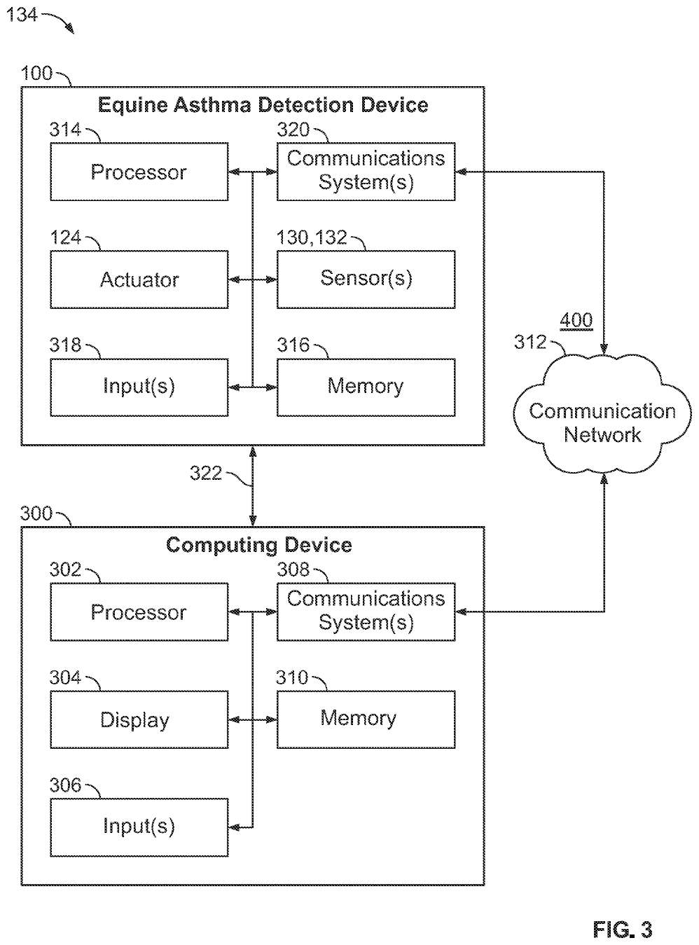

As will be described, the interrupt assembly 120, including the sensors 130, 132, and the actuator 124 may be connected to a control system 134. In some examples, the power source can be connected to the control system. For instance, a power source can receive a signal from a control system to close a shutter of an interrupt assembly and can further receive instructions from the control system dictating a total interrupt interval. In some cases, the control system 134 can communicate with the interrupt assembly 120 to activate a switch (not shown) to selectively enable power from the power source 150 to drive a desired operation of the shutter 126. As will be described with respect to FIG. 3, the control system may take any of a variety of forms. In one non-limiting example, the control system 134 may include a computing device 300. As will be described, the computing device can be configured to control the equine asthma detection device 100 to implement a process for determining equine asthma.

As illustrated in FIG. 3, the computing device 300 can include a processor 302, a display 304, one or more inputs 306, one or more communication systems 308, and/or memory 310. In some configurations, the processor 302 can be any suitable hardware processor or combination of processors, such as a central processing unit (CPU), a graphics processing unit (GPU), an application specific integrated circuit (ASIC), a field-programmable gate array (FPGA), and the like. In some configurations, the display 304 can include any suitable display device, such as a computer monitor, a touchscreen, a television, and the like. In some configurations, the inputs 306 can include suitable input devices and/or sensors that can be used to receive user input, such as a keyboard, a mouse, a touchscreen, a microphone, a camera, and the like. The computing device 300 may be formed as a computer, phone, tablet, or other computing or control device.

In some configurations, the communications systems 308 can include suitable hardware, firmware, and/or software for communicating information over a communication network 312 and/or any other suitable communication networks. For example, communications systems 308 can include one or more transceivers, one or more communication chips and/or chip sets, and the like. In a more particular example, the communications systems 308 can include hardware, firmware and/or software that can be used to establish a Wi-Fi connection, a Bluetooth connection, a cellular connection, an Ethernet connection, and the like.

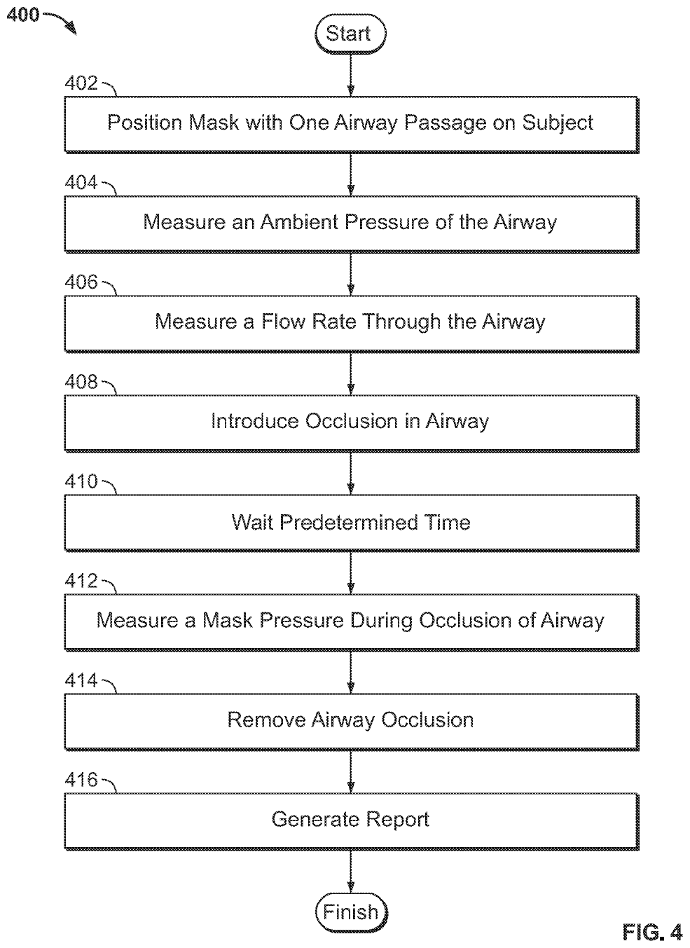

In some configurations, the memory 310 can include a suitable storage device or devices that can be used to store instructions, values, and the like, that can be used, for example, by processor 302 to calculate an airway resistance of a horse or generate graphics representing the measurements of the sensors 130, 132 and display the information or graphics to the display 304. The memory 310 can include any suitable volatile memory, non-volatile memory, storage, or any suitable combination thereof. For example, the memory 310 can include random access memory (RAM), read-only memory (ROM), electronically-erasable programmable read-only memory (EEPROM), one or more flash drives, one or more hard disks, one or more solid state drives, one or more optical drives, and the like. In some configurations, the memory 310 can have encoded thereon a computer program for controlling operation of the computing device 300. For example, in such configurations, the processor 302 can execute at least a portion of the computer program to receive inputs from a graphical user interface for setting a value of the system, for example, an occlusion duration, or units to be displayed. As another example, the processor 302 can execute at least a portion of the computer program to implement the process that will be described with respect to FIG. 4 for obtaining measurements from the sensors 130, 132 and producing a signal to control operation of the actuator 124. Commercially available software may be provided in memory 310 and implemented by processor 302 to perform some functions of the system. For example, in some configurations, a commercial software system (IOX2 measurement, Emka Technologies, Norfolk VA and Paris, France) can be used to visualize the waveforms and store flow measurements. However, other software and systems can be used for visualization of waveforms.

As further illustrated in FIG. 3, the equine asthma detection device 100 can include computing elements which, in some configurations, can allow control and/or processing to be performed by the equine asthma detection device 100, even without the computing device 300 in some situations, or with a more simplified computing device 300 in some situations. For example, the device 100 can include a processor 314, a memory 316, inputs 318, and communication system 320, which can be generally similar to, processor 302, memory 310, inputs 306, and communication system 308 respectively, such that the descriptions of the latter are applicable to the formed. The input(s) 318 can includes a power button, a dial, buttons, touch screens, or other user interfaces that allow a user to utilize the device 100. For example, the input(s) 318 may allow a user to set an occlusion time, or to control a mode of the sensors 130, 132. An input 318 can also control the function of the shutter 226 of FIGS. 1 and 2, and accordingly, a switch can be provided to allow an operator to manually move the shutter 226 between the first and second positions 127, 128. In other configurations, the device 100 could further include displays (e.g., LCD displays) for displaying a measurement of the sensors, or a calculated resistance. In some configurations, the computing device 300 may implement the method that will be described with respect to FIG. 4 by receiving measurements from the sensors 130, 132, and sending a signal to the actuator 124 to selectively move the shutter 126 between the first, or open position, and the second, or closed position.

In the illustrated configuration, the equine asthma detection device 100 and the computing device 300 can communicate over communication network 312 or could alternatively be operably connected using a physical connection 322, which can be a wire or a plurality of wires. In some configurations, each of the pressure sensors 130 and/or flow sensors 132 can include communication systems that enable the sensors to individually communicate with the computing device 300 directly. In some configurations, the actuator can also communicate with the computing device 300 directly. In some configurations, an equine asthma detecting device 100 may not have computing elements 314, 316, or 320, which may allow the device 100 to be more lightweight, cheaper, and/or portable.

The device 100 described above is designed to operate based on different physiological principles than conventional devices and methods for detecting an asthma of a horse. For example, though measurement of alveolar pressure can be invasive and impractical to perform directly, the alveolar pressure can be observed indirectly when an equilibrium is achieved between an alveolar pressure, and pressure in a breathing compartment, such as the chamber 108. The mask 102 can be provided to channel an airflow of the wearer's breath and provide a chamber 108 that can be fluidly isolated from ambient air 118. When an interruption or occlusion is introduced in the airway by the shutter 126, isolating the breathing chamber 108 of the mask 102 from ambient air 118, the chamber 108 is only in fluid communication with the lungs of the horse 106. Thus, after a period of time, the pressure in the breathing chamber 108 can reach equilibrium with the alveolar pressure and measuring the pressure in the breathing chamber 108 can provide a measurement of alveolar pressure. In this configuration, then, resistance can be calculated based on the difference in the pressure of the ambient air 118 and the pressure within the mask 108, and the change in volume can be obtained by measuring a flow rate through the airway immediately prior to introduction of the occlusion by the shutter 126, as given by:

$$Resistance = \frac{( ambient air pressure - mask air pressure )}{air flow rate immediately before occlusion} . \qquad ( 2 )$$

As described above, the shutter is designed to close sufficiently quickly to not allow a loss of air from the mask chamber that would be sufficient to undermine the determination of equine asthma. In one non-limiting example, consider that the average horse may have a resting lung (tidal) volume of 8.0 liters, and a transient, non-forced peak flow of 4.5 l/s. In this non-limiting example, the maximum air lost from the system during a closure time of 20 ms would be 90 mls, which is a 1% loss of total breathing volume before complete closure. If the speed of the shutter moving to fully closure were 50 ms, the loss would be 225 mls, which is a 2.8% loss of total breathing volume. Accurate measurements of resistance can be obtained from an equine asthma detection device with a loss of 5% or less of a total breathing volume for a horse. Notably, the above, non-limiting examples calculate a “maximum” air loss by assuming that airflow during the time to complete closure is unimpeded. However, in practice, airflow begins to be restricted as the shutter closes. Thus, in actual studies the losses are less than 1% or 2.8%, respectively, because those percentages were calculated as “maximum” air losses that do not account for partial airflow restriction as the shutter moves to closure.

In accordance with one, non-limiting example, the shutter may remain completely closed for 100-200 ms, which, using the above-described device 100, is designed to not allow the horse 106 to even notice that the airway was obstructed. Thus, as described, the interrupt assembly 120 may be designed to close the shutter 126 in, for example, less than 50 ms, or less than 20 ms, or less than 10 ms, or another speed that the horse 106 cannot register, and to stay closed for 50-200 msec, during which time equilibration of mask pressure with alveolar pressure occurs, and the horse does not notice the occlusion. As the device 100 and the method of using the device 100 does not require that a subject perform different breathing procedures in accordance with instructions, it can be implemented to detect equine asthma.

Thus, referring to FIG. 4, the steps of one non-limiting example of a method 400 of using the above-described device 100 to perform an interrupter technique to obtain a resistance measurement of a horse is provided. At block 402, a mask may be positioned on the horse. The mask can provide a seal, preventing airflow in any direction but through the airway passage provided. Positioning the mask on the horse can involve inserting a muzzle of the horse through the mask, and placing a strap (e.g., strap 111 shown in FIG. 1) around a head of the horse to maintain the mask in place. In some cases, a handler of the horse supports the mask in place on the horse during operation of the test.

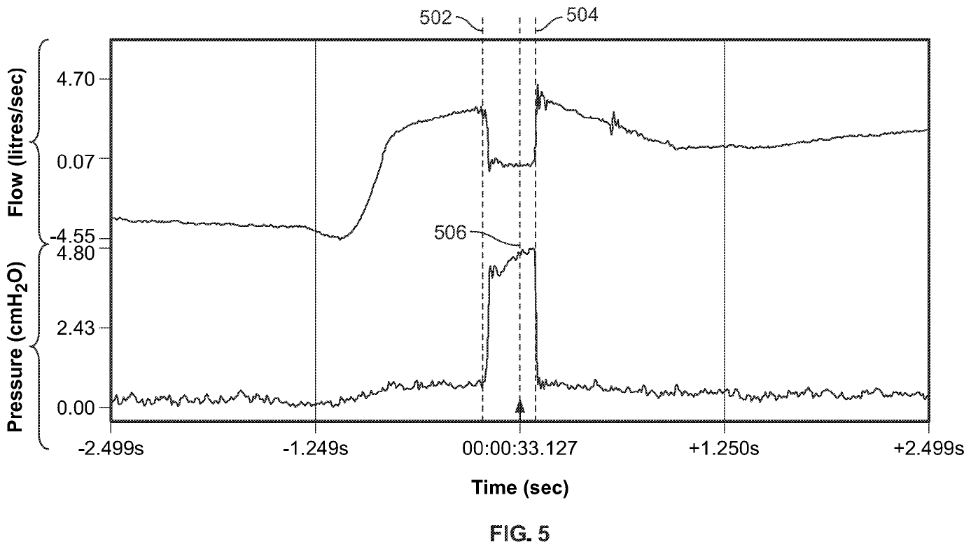

At block 404, an ambient pressure can be sensed, using a pressure sensor within the airway, as further described below. In some cases, an ambient air pressure can be measure at a given point in time (e.g., immediately before introduction of an occlusion in the airway, or at time 502 illustrated in FIG. 5). In some cases, the method can obtain multiple ambient air pressure measurements, including during inspiration and expiration of the horse, and can determine an average ambient air temperature for use in a resistance calculation. In some cases, the

At block 406, which can be performed in parallel or serially with the steps in block 404, a flow rate is measured through the airways (e.g., liters per second of air flowing through the airway). Subsequent to measuring the flow rate at block 406, an occlusion can be introduced into the airway at block 408. This occlusion can effectively seal off fluid communication between the air in the mask and ambient air, stopping the flow of air. Once the occlusion has been introduced, a wait time can be implemented in block 410 (e.g., the time interval between time 506 and time 502 shown in FIG. 5). This time can allow the pressure of the air within the mask to reach an equilibrium with the alveolar pressure of the subject. In some examples, the wait time can be approximately 150 ms. In some examples, the wait time can be about 170 ms, or between about 100 ms and 200 ms.

FIG. 5 is a graph showing a change of pressure and flow rate of air through a mask as a function of time in response to an occlusion introduced in the airway of a horse (e.g., using device 100). As shown, an occlusion is introduced at a first time 502 (e.g., by moving shutter 126 to the second position 128 illustrated in FIG. 1). A pressure measured at time 502 can be used as the ambient air pressure value of equation (2), and a flow rate of air through the mask measure at time 502 can be used as the airflow rate immediately before occlusion shown in equation (2). As shown a pressure can increase in response to the occlusion at the first time 502, and a flow rate can begin to decrease at the first time 502. There is a delay in the time between when an occlusion is introduced at the first time 502 and when equilibrium in flow rate is reached at time 506. According to the physiologically well-understood breath interruption method, the lungs must be allowed to elastically react to the pressure build after breath occlusion before an accurate pressure reading can be measured, and the reaction may produce a sinusoidal phenomenon in a pressure of the mask. For example, the horse (e.g., lungs of the horse) can continue breathing motions even as the horse is unable to breathe during the interruption, and the breathing motions can at least partially produce the sinusoidal phenomenon. The sinusoidal phenomenon can end, on average, at about 150 ms for horses, before the pressure differential begins to increase. At this point as well, the alveolar pressure has sufficient time to equalize with pressure inside the mask chamber 108. Thus, a time between the occlusion at time 502, and a steady state pressure measured at time 506 can be about 150 ms. In some cases, an equine asthma detection device (e.g., device 100 shown in FIG. 1) can implement a delay of 170 ms before measuring a pressure of the mask enclosure (e.g., a time between occlusion time 502 and the measurement time 506 can be 170 ms). A pressure obtained at time 506 (e.g., at a set time interval after time 502) can be used as the mask air pressure in equation (2), and the combination of the pressure and flow measurements obtained at time 502, and the pressure measurement obtained at time 506 can be used to obtain a Resistance measurement, as shown in equation (2). In other example, other time intervals between an occlusion and a pressure measurement of a mask can be used, including as can be adapted to horses of different sizes, ages, breeds, etc.

Bases for selecting the length of an occlusion can be the time it takes for equilibrium to be reached, and/or a tolerance of the subject for the airway restriction. In testing, for example, it was found that horses could tolerate occlusions of a duration of up to 200 ms. Longer restrictions may produce a spooking or other adverse reaction of the horse, and therefore, the occlusion time may be kept under 200 ms or another suitable threshold to reach equilibrium but not extend substantially longer. In some cases, the length of an occlusion can be set to 170 ms from when an operator of the system initiates the occlusion. After the predetermined time, once equilibrium has been reached, at block 412, pressure is measured within the mask, which can serve as a proxy for the alveolar pressure. Once the pressure measurement has been taken, the occlusion can be removed at block 414, allowing the subject to breathe freely through the airway (e.g., at time 504 shown in FIG. 5). At block 416, a report indicating an equine asthma condition of the horse can be generated. For example, an airway resistance can be calculated based on the measure flow and pressure values, in accordance with equation 2. This resistance can then be evaluated, for example, against a table, database, or chart to then generate the report indicating whether a horse is suffering from asthma. Thus, the process 400 can be used to detect an asthma in a horse. In some configurations, the process 400 can be repeated multiple times to receive multiple resistance measurements.

### EXAMPLES

The following tests were performed using the example methods and apparatuses described above, hereinafter referred to as “EquiRint” and conventional methods for detecting equine asthma, with the results of the tests shown in the tables provided below.

EquiRint v. EB/P:

In a test comparing the effectiveness of an EquiRint device (e.g., device 100 shown in FIG. 1) with EB/P equine asthma detection, ten horses with mild equine asthma were tested at baseline, with the measurement variable R_{INT} obtained in cmH2O/l/s for the EquiRint and R_{L}cmH2O/l/s for EB/P. The horses were then challenged with histamine. Histamine is a substance that causes temporary narrowing of the airways and is used as a clinical test of asthma in horse (airway hyperreactivity, or AHR). Response to histamine is measured as mg/ml of histamine necessary to elicit a 75% increase in airway resistance, and termed PC75R_{INT} (in the case of measurement by the interrupter technique of the EquiRint device) or PC75R_{L} (in the case of measurement with esophageal balloon/pneumotachography).

Further, six horses with severe equine asthma were tested at baseline in the manner described above. These six horses were subsequently challenged with albuterol, a drug that causes dilation of airways that are constricted due to disease. Response to albuterol is measured as percent decrease in resistance measured by each device.

Additionally, six control horses (horses without history or clinical signs of respiratory disease) were tested at baseline with EquiRint v EB/P at one hour, one week, and one-month intervals to determine stability of measurements.

Paired t-tests were performed to determine precision of the R_{INT} measurement at baseline when compared to the measurement obtained via EB/P, which is currently a gold standard for measurement of pulmonary resistance in horses. As shown in Table 1, the correlation between the 2 measurements is high, and there is no statistically significant difference between the two measurements.

TABLE 1
t-Test: Paired Two Sample for Means at Baseline

| | EquiRint | EB/P |
|---|---|---|
| Mean Resistance (cmH2O/l/s) | 0.41 | 0.394 |
| Observations | 10 | 10 |
| Pearson Correlation | 0.92361184 | |
| Hypothesized Mean Difference | 0.05 | |
| df | 9 | |
| t Critical two-tail | 2.26215716 | |

Chi-square tests were further performed to determine if the EquiRint device and the EB/P measurements identified similar hyperreactivity, with hyperactivity being defined as PC75R_{INT} or PC75R_{L}>6 mg/ml of histamine. Out of ten horses, RINT diagnosed five horses as having airway reactivity, and five as non-reactive. Using EB/P, one of the reactive horses would have been classified as non-reactive; thus, EquiRint was more sensitive in detection of airway narrowing than EB/P.

Paired t-tests were performed to compare baseline measurements and response to albuterol bronchodilation tests using EquiRint v. EB/P in six horses.

TABLE 2
t-Test: Paired Two Sample for Means - Baseline
Measurements - Severe Airway Obstruction

| | EquiRint (R_{INT}) | EB/P (R_{L}) |
|---|---|---|
| Mean resistance (in cmH2O/l/s) | 2.19 | 1.99 |
| Observations | 6 | 6 |
| Pearson Correlation | 0.92 | |
| Hypothesized Mean Difference | 0.05 | |
| df | 5 | |
| t Critical two-tail | 4.30265273 | |

As expected, the airway resistance in these severely affected horses was approximately four times as high as the horses with mild disease. The correlation between the two methods was high (e.g., the correlation as measured with a Pearson correlation was 0.92, as shown in Table 2), indicating excellent comparability between the test performed with the EquiRint device, and the test performed using EB/P.

TABLE 3
t-Test: Paired Two Sample for Means -
Bronchodilator Challenge with Albuterol

| | EquiRint | EB/P |
|---|---|---|
| | (% change) | (% change) |
| Mean | 24.69 | 22.65 |
| Observations | 6 | 6 |
| Pearson Correlation | 0.885 | |
| Hypothesized Mean Difference | 0.05 | |
| df | 7 | |
| t Critical two-tail | 2.3646243 | |

In the measurements of the response of the six horses with severe equine asthma to a challenge with Albuterol (e.g., shown in table 3), the measurements using EquiRint, and EB/P produced similar results. For example, similar to baseline measurements, there was a strong correlation (0.856) between percentage decrease in airway resistance using the EquiRint as compared with EB/P.

EquiRint v. FOM:

Further testing was performed to compare a performance of the EquiRint device in measuring pulmonary resistance compared to measurements obtained using FOM. In a first test, ten horses with moderate equine asthma were tested at baseline, with the measurement variable R_{INT} in cmH2O/l/s for the EquiRint and RRs in cmH2O/l/s for FOM, and the results are provided below in Table 4.

TABLE 4
t-Test: Paired Two Sample for Means - Baseline EquiRint v FOM

| | EquiRint (R_{INT}) | FOM (R_{RS}) |
|---|---|---|
| Mean | 0.505 | 0.553 |
| Variance | 0.0255833 | 0.03529 |
| Observations | 10 | 10 |
| Pearson Correlation | 0.84 | |
| Hypothesized Mean Difference | 0.05 | |
| df | 9 | |
| t Critical two-tail | 2.2621572 | |

Resistance was mildly higher in these horses with more significant disease. Resistance measured with FOM (RRs) was consistently slightly higher than that measured with EquiRint (R_{INT}). As FOM measures the resistance of the entire respiratory system, including the chest wall, this was an expected finding. The correlation was high (0.84), again showing good performance but not exact equivalence for 2 tests measuring slightly different physiological outcomes.

Eight horses with mild equine asthma were challenged with histamine, a substance that causes temporary narrowing of the airways and is used as a clinical test of asthma in horse (airway hyperreactivity, or AHR), measured as mg/ml of histamine necessary to elicit a 75% increase in airway resistance. A comparison of the measurements of resistance using EquiRint and FOM are provided below in Table 5. Both the EquiRint and FOM tests assigned the same five horses out of eight to ‘reactive’ v. ‘non-reactive’ categories.

TABLE 5
t-Test: Paired Two Sample for Means - AHR in Horses
with Moderate Equine Asthma EquiRint v FOM

| | EquiRint (R_{INT}) | FOM (R_{RS}) |
|---|---|---|
| Mean - mg/ml histamine | 6.79 | 7.96 |
| Observations | 8 | 8 |
| Pearson Correlation | 0.9623079 | |
| Hypothesized Mean Difference | 0.05 | |
| df | 7 | |
| t Critical two-tail | 2.3646243 | |

No horses with severe asthma were tested with FOM, as the increased work of breathing associated with this disease interferes with the measurements.

The previous description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

In some implementations, devices or systems disclosed herein can be utilized, manufactured, installed, etc. using methods embodying aspects of the invention. Correspondingly, any description herein of particular features, capabilities, or intended purposes of a device or system is generally intended to include disclosure of a method of using such devices for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated or limited, discussion herein of any method of manufacturing or using for a particular device or system, including installing the device or system, is intended to inherently include disclosure, as embodiments of the invention, of the utilized features and implemented capabilities of such device or system.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” For example, a list of “one of A, B, or C” indicates options of: A, but not B and C; B, but not A and C; and C, but not A and B. A list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of A, one or more of B, and one or more of C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C.

In some implementations, devices or systems disclosed herein can be utilized, manufactured, installed, etc. using methods embodying aspects of the invention. Correspondingly, any description herein of particular features, capabilities, or intended purposes of a device or system is generally intended to include disclosure of a method of using such devices for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated or limited, discussion herein of any method of manufacturing or using for a particular device or system, including installing the device or system, is intended to inherently include disclosure, as embodiments of the invention, of the utilized features and implemented capabilities of such device or system.

In some embodiments, aspects of the invention, including computerized implementations of methods according to the invention, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, embodiments of the invention can be implemented as a set of instructions, tangibly embodied on a non-transitory computer-readable media, such that a processor device can implement the instructions based upon reading the instructions from the computer-readable media. Some embodiments of the invention can include (or utilize) a control device such as an automation device, a special purpose or general-purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.). In some embodiments, a control device can include a centralized hub controller that receives, processes and (re)transmits control signals and other data to and from other distributed control devices (e.g., an engine controller, an implement controller, a drive controller, etc.), including as part of a hub-and-spoke architecture or otherwise.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the invention, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the invention. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” “block,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

Also as used herein, unless otherwise limited or defined, the terms “about,” “substantially,” and “approximately” refer to a range of values ±5% of the numeric value that the term precedes. As a default the terms “about” and “approximately” are inclusive to the endpoints of the relevant range, but disclosure of ranges exclusive to the endpoints is also intended.

Also as used herein, unless otherwise limited or defined, “integral” and derivatives thereof (e.g., “integrally”) describe elements that are manufacture as a single piece without fasteners, adhesive, or the like to secure separate components together. For example, an element stamped as a single-piece component from a single piece of sheet metal, without rivets, screws, or adhesive to hold separately formed pieces together is an integral (and integrally formed) element. In contrast, an element formed from multiple pieces that are separately formed initially then later connected together, is not an integral (or integrally formed) element.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

1. A system for detecting equine asthma in a horse, the system comprising:
a mask configured to enclose a portion of a muzzle of a horse;
an airflow path from the mask;
a shutter configured to obstruct the airflow path from the mask;
at least one sensor configured to measure at least one of air pressure or airflow before the shutter obstructs the airflow path and to measure at least one of air pressure or airflow after the shutter obstructs the airflow path;
a processor configured to:
receive feedback from the at least one sensor;
determine a pressure in the mask after the shutter obstructs the airflow path from the mask; and
generate a report indicating an equine asthma condition of the horse.

2. The system of claim 1, wherein the processor is further configured to determine a resistance using the feedback from the at least one sensor and include one of the resistance or a comparison of the resistance against an equine resistance value to generate the report indicating the equine asthma condition of the horse.

3. The system of claim 2, wherein the process is further configured to calculate resistance as:
$$\frac{( ambient air pressure - mask air pressure )}{air flow rate immediately before occlusion} .$$

4. The system of claim 1 wherein the airflow path is defined by a tube extending from the mask, and wherein the tube is positioned along a top portion of the mask, or wherein the tube is configured to be positioned proximate to a nose of the horse when the mask encloses the portion of the muzzle of the horse.

5. The system of claim 4, wherein the at least one sensor includes a first sensor configured to measure an airflow through the tube and a second sensor configured to measure an air pressure within the tube.

6. The system of claim 5, wherein the shutter is positioned between the first sensor and the second sensor.

7. The system of claim 1, further comprising an actuator configured to move the shutter between a first position and a second position, wherein, in the first position, the shutter does not obstruct the airflow path, and wherein, in the second position, the shutter obstructs the airflow path.

8. The system of claim 7, further comprising a power source configured to power the actuator, wherein, in response to a power signal from the power source, the actuator moves the shutter from the first position to the second position.

9. The system of claim 8, wherein the power source comprises at least one cannister containing a compressed air.

10. The system of claim 1, wherein the processor is further configured to:
determine an ambient pressure before the shutter obstructs the airflow path from the mask;
determine an airflow rate through the airflow path before the shutter obstructs the airflow path from the mask;
implement a wait interval between the measurement of the ambient pressure and the measurement of the pressure in the mask.

11. The system of claim 10, wherein the wait interval is at least 150 ms.

12. A system for detecting equine asthma in a horse, the system comprising:
a mask including a top portion to partially receive a nose of a horse and a bottom portion to partially receive a mouth of the horse, the mask defining a mask chamber;
a tube, the tube protruding from the top portion of the mask, the tube including a first opening in fluid communication with the mask chamber and a second opening in fluid communication with an ambient air;
a pressure sensor disposed at a first position within the tube, the pressure sensor being configured to measure an air pressure within the tube;
a flowmeter disposed at a second position within the tube, the flowmeter being configured to measure an air flow through the tube;
an interrupt assembly including an actuator and a shutter, the actuator configured to move the shutter between:
a first position, in which the shutter does not extend into the tube, and
a second position in which the shutter obstructs an airflow path through the tube, wherein, in the second position, the shutter is positioned between the pressure sensor and the flowmeter; and
a processor configured to:
receive, from the flowmeter, a first flow rate measurement,
receive, from the pressure sensor, a first pressure measurement,
determine, based at least in part on the first flow rate measurement and the first pressure measurement, an equine asthma condition of the horse, and
output to one of a display or a memory, the equine asthma condition.

13. The system of claim 12, wherein the first pressure measurement is received at a first time, wherein the processor is further configured to:
receive, at a second time, from the pressure sensor, a second pressure measurement,
wherein the determination of the equine asthma condition is based, at least in part on the second pressure measurement.

14. The system of claim 13, wherein, at the first time, the shutter is in the first position, and at the second time, the shutter is in the second position.

15. The system of claim 14, wherein the equine asthma condition includes a resistance value, wherein the resistance value comprises a difference between the first pressure measurement and the second pressure measurement divided by the first flow rate measurement.

16. The system of claim 13, wherein a time interval between the first pressure measurement and the second pressure measurement is at least 150 ms.

17. The system of claim 12, wherein the actuator is pneumatically activated in response to a compressed air received from a compressed air cannister.

18. A method of determining whether a horse has equine asthma, the method including steps comprising:
a) engaging the horse with a system that provides a single airflow passage for the horse as the horse breathes;
b) measuring a rate of air flow through the single airflow passage and an ambient air pressure at a first time;
c) obstructing the single airflow passage as the horse continues breathing motions;
d) measuring a pressure about a muzzle of the horse at a second time while the single airflow passage is obstructed and the horse continues breathing motions;
e) analyzing the pressure about the muzzle of the horse, the rate of air flow, or the ambient air pressure to determine that the pressure about a muzzle of the horse while the single airflow passage is obstructed and the horse continues breathing motions is indicative of equine asthma; and
d) generating a report indicating that the horse is suffering from equine asthma.

19. The method of claim 18, wherein steps b)-e) are performed automatically under control of a system including a processor.

20. The method of claim 19, wherein the processor is configured to implement a wait interval between the first time and the second time, wherein the wait interval is at least 150 ms.
